(19) United States
(12) Patent Application Publication
(10) Pub. No.: US 2025/0114134 A1
(43) Pub. Date: Apr. 10, 2025

(54) ARTHROSCOPIC WASHER EXTRACTOR

(71) Applicant: Shukla Medical, St. Petersburg, FL (US)

(72) Inventors: Malcolm DeBaun, St. Petersburg, FL (US); Austin Ryan Stroh, Lutz, FL (US)

(73) Assignee: Shukla Medical, St. Petersburg, FL (US)

(21) Appl. No.: 18/904,353

(22) Filed: Oct. 2, 2024

Related U.S. Application Data
(60) Provisional application No. 63/588,179, filed on Oct. 5, 2023.

(51) Int. Cl. A61B 17/92; A61B 17/00; A61B 17/29

(52) U.S. Cl. CPC A61B 17/921; A61B 2017/00296; A61B 2017/2932

(57) ABSTRACT

An arthroscopic washer extractor including a handle, an actuator pivotable relative to the handle and movable between a neutral position and a retracted position, a shaft operatively connected to the actuator, and a pair of jaws at least one of which is operatively connected to the shaft, wherein each of the pair of jaws includes a radially outwardly extending flange, and wherein when the actuator is in the retracted position the pair of jaws move to an expanded state operable to engage an arthroscopic washer.

### CROSS-REFERENCE TO RELATED APPLICATION(S)

This application claims the benefit under 35 U.S.C. § 119(e) to U.S. Provisional Application No. 63/588,179, filed Oct. 5, 2023, and entitled “Arthroscopic Washer Grasper,” the disclosure of which is hereby incorporated by reference in its entirety for all purposes.

### FIELD OF THE INVENTION

The present invention relates to devices and methods for arthroscopically removing an implant, e.g., a washer, from a patient.

### BACKGROUND OF THE DISCLOSURE

At times, upon attempting to arthroscopically remove surgically placed screws and washers from a patient, a washer does not come out with the screw. Because of the flat profile of a washer, the retained washer is often difficult to grasp and remove. The subject disclosure provides a device and method to remove a retained washer in a time efficient manner that minimizes trauma to the tissues surrounding the washer.

### SUMMARY OF THE DISCLOSURE

According to an exemplary embodiment, the subject disclosure provides an arthroscopic washer extractor comprising: a handle; an actuator pivotable relative to the handle and movable between a neutral position and an operative position; a shaft operatively connected to the actuator; a pair of jaws at least one of which is operatively connected to the shaft, wherein each of the pair of jaws includes a radially outwardly extending flange and wherein, wherein the actuator is configured to move the pair of jaws to an open state.

According to an aspect, the arthroscopic washer extractor further comprises a biasing member operatively engaged with the actuator and biasing the actuator towards the neutral position. According to another aspect the biasing member can be a spring, a coil spring, a tension spring or an elastomer.

According to an aspect, the arthroscopic washer extractor further comprises a pair of levers pivotably connected to a distal end of the shaft and pivotably connected to the pair of jaws. According to another aspect, the distal end of the shaft is provided with a pair of lobes pivotably connected to the pair of levers.

According to an aspect, the handle includes an elongated frame. According to another aspect, the elongated frame includes a pair of spaced-apart arms. According to another aspect, the elongated frame includes a support beam extending between and joined to the pair of spaced-apart arms. According to another aspect, the support beam forms boundaries of a first recess and a second recess provided in the elongated frame. According to another aspect, the first recess is configured to receive and pivotably mount the actuator to a proximal end of the elongated frame. According to another aspect, the second recess is configured to receive and pivotably mount the pair of jaws to a distal end of the elongated frame. According to another aspect, the support beam has a notched nose at a distal end thereof. According to another aspect, a distal end of the shaft includes a substantially U-shaped turn for receiving the notched nose of the support beam.

According to an aspect, an arm of the actuator includes a first through hole for receiving a proximal end of the biasing member. According to another aspect, the arm of the actuator includes a second through hole for pivotably connecting with an elongated frame of the handle. According to another aspect, the arm of the actuator includes a third through hole for pivotably connecting with the shaft.

According to an aspect, one of the pair of jaws is integrally connected to the elongated frame. According to another aspect, the other of the pair of jaws is pivotably connected to the shaft. According to another aspect, the other of the pair of jaws is pivotably connected to the elongated frame.

According to an aspect, the handle includes an elongated frame, and the elongated frame and the shaft define a cannulation. According to another aspect, the cannulation is formed by opposed elongated grooves provided in the elongated frame and the shaft. According to another aspect, the opposed elongated grooves are concave.

According to another exemplary embodiment, the subject disclosure provides a method for grasping and removing a washer from a patient comprising introducing an arthroscopic washer extractor according to the subject disclosure into a patient, inserting the pair of jaws through a hole of a washer at least until the radially outwardly extending flanges of the jaws pass through the washer, actuating the actuator to open the jaws whereby the radially outwardly extending flanges engages a distal face of the washer, and pulling the arthroscopic washer extractor back toward a user with the opposing jaws in an open position.

### BRIEF DESCRIPTION OF THE DRAWINGS

The foregoing summary, as well as the following detailed description of the exemplary embodiments of the subject disclosure, will be better understood when read in conjunction with the appended drawings. For the purpose of illustration, there are shown in the drawings exemplary embodiments of the subject disclosure. It should be understood, however, that the subject disclosure is not limited to the precise arrangements and instrumentalities shown.

FIG. 1 is a side view of an arthroscopic washer extractor in accordance with a first exemplary embodiment of the subject disclosure;

FIG. 2 is a partial cross sectional view of the washer extractor of FIG. 1;

FIG. 3 is an exploded side view of the washer extractor of FIG. 1;

FIG. 4 is a side view of an exemplary handle and elongated frame of the washer extractor of FIG. 1;

FIG. 5 is a top perspective view of an exemplary handle and elongated frame of the washer extractor of FIG. 1;

FIG. 6 is a perspective view of an exemplary actuator of the washer extractor of FIG. 1;

FIG. 7 is a perspective view of an exemplary shaft of the washer extractor of FIG. 1;

FIG. 8 is a perspective view of an exemplary jaw of the washer extractor of FIG. 1;

FIG. 9 is a perspective view of an exemplary lever of the washer extractor of FIG. 1;

FIGS. 10A-10C illustrate sequential steps for grasping and extracting a washer using an exemplary arthroscopic washer extractor of the subject disclosure.

FIG. 11 is a perspective view of a distal end of an elongated frame of the washer extractor of FIG. 1;

FIG. 12 is a perspective view of exemplary jaws and levers of the washer extractor of FIG. 1;

FIG. 13 is a side view of an arthroscopic washer extractor in accordance with a further exemplary embodiment of the subject disclosure;

FIG. 14 is a distal end view of the arthroscopic washer extractor of FIG. 13 as seen from line 14-14 of FIG. 13;

FIG. 15 is a side view of an exemplary handle and elongated frame of the washer extractor of FIG. 13;

FIG. 16 is a top plan view of the handle and elongated frame of FIG. 15;

FIG. 17 is a top perspective view of the handle and elongated frame of FIG. 15;

FIG. 18 is a side view of an exemplary shaft of the of the washer extractor of FIG. 13;

FIG. 19 is a bottom perspective view of an exemplary shaft of FIG. 18;

FIG. 20 is a side view of an exemplary actuator of the washer extractor of FIG. 13;

FIG. 21 is a perspective view the actuator of FIG. 20;

FIG. 22 is a perspective view of a movable jaw of the washer extractor of FIG. 13; and

FIG. 23 is a distal end view of the movable jaw of FIG. 22.

### DETAILED DESCRIPTION

Reference will now be made in detail to the various exemplary examples of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages, and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the subject disclosure.

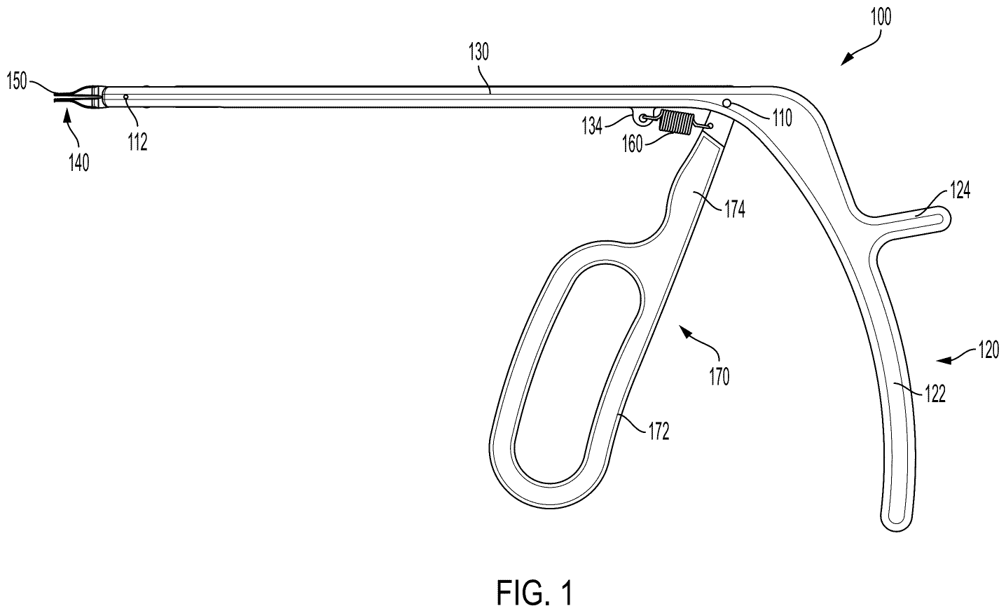

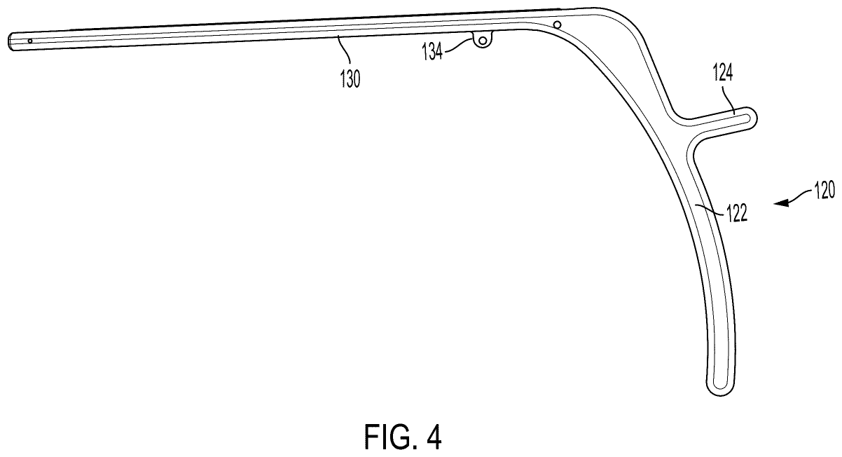

Referring to the drawings, FIGS. 1-3 illustrate an exemplary embodiment of an arthroscopic washer extractor 100 that comprises a handle 120, an actuator 170, a shaft 190, and a pair of jaws 140. The handle 120 is configured as best shown in FIGS. 1-5. The handle 120 includes an elongated curved gripping portion 122 having a stop 124 extending posteriorly therefrom about an upper end of the gripping portion. Specifically, the stop extends about a portion one-third down from an uppermost end of the handle, e.g., about 1 to 2 inches from its uppermost end or about 2 to 4 inches from its bottom most end. The stop 124 has an overall length of about 0.5 to 1.5 inches and extends posteriorly at an angle of about 10 degrees. The handle is curved, e.g., having an arc radius of about 4 to 8 inches. The gripping portion 122 is sized to have an overall length sufficient for a palm of a person's hand to grasp, e.g., about 3 to 6 inches in length.

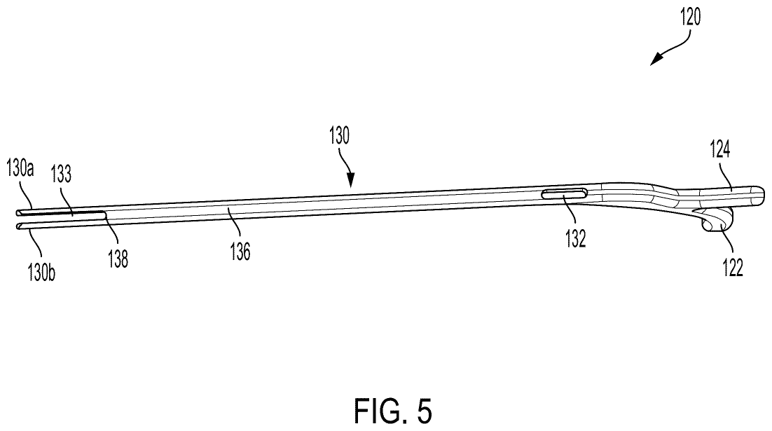

The handle 120 includes an elongated frame 130 extending therefrom. The elongated frame has a longitudinal axis extending substantially transverse to a longitudinal axis of the curved gripping portion 122. As shown in FIGS. 1-4, a proximal portion of the elongated frame adjacent to the elongated gripping portion includes a tab or protrusion 134 having a hole to receive and connect to one end of a biasing member 160. As shown in FIG. 5, the elongated frame 130 includes a pair of spaced-apart arms 130a, 130b. The spaced apart arms 130a, 130b are spaced sufficiently to receive the shaft 190 therein, as further discussed below.

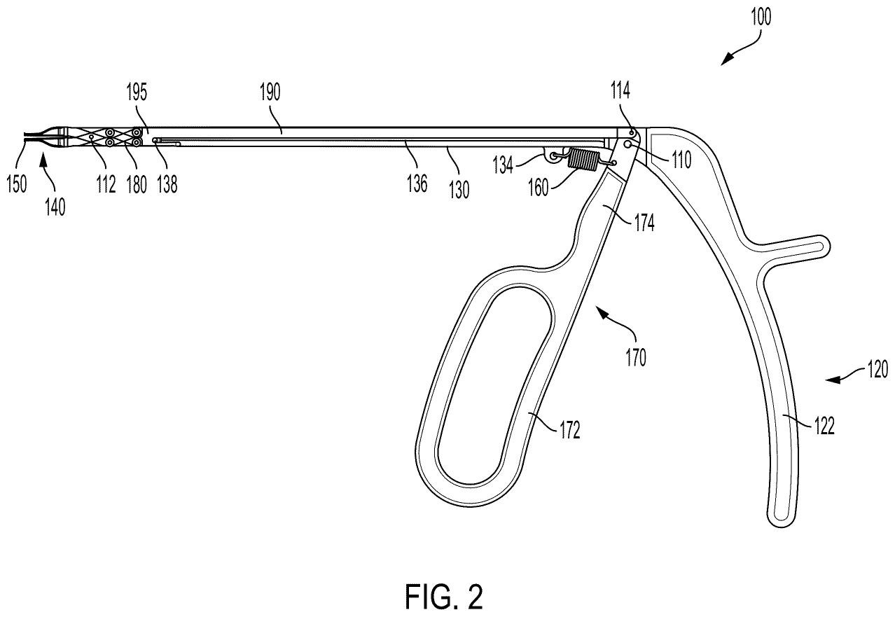

As shown in FIG. 5, the elongated frame 130 also includes a first recess 132 about its proximal or posterior end for receiving a first pivot 110 (FIGS. 1 and 2) therein and a second recess 133 about its distal or anterior end for receiving a second pivot 112 (FIGS. 1 and 2) therein. The first pivot and second pivot can be, e.g., a pin or rivet. By way of the first pivot 110, the first recess 132 is configured to receive and pivotably mount the actuator 170 to the proximal end of the elongated frame 130 of the handle 120. By way of the second pivot 112, the second recess 133 is configured to receive and pivotably mount the pair of jaws 140 to the distal end of the elongated frame 130 of the handle 120.

Referring still to FIG. 5, a support beam 136 extends between and is joined to the pair of spaced-apart arms 130a, 130b to provide structural support to the elongated frame 130 and form boundaries of the first recess 132 and the second recess 133. Referring to FIGS. 2, 5 and 13, a distal end of the support beam 136 includes a notched nose 138 constructed and arranged to be received within a U-shaped turn 195 (FIGS. 2, 8 and 13) disposed about a distal end of the shaft 190. The notched nose 138 is sized sufficiently to allow longitudinal movement of the shaft 190 relative thereto thereby allowing full range of motion of levers 180, described below, between expanded and contracted positions.

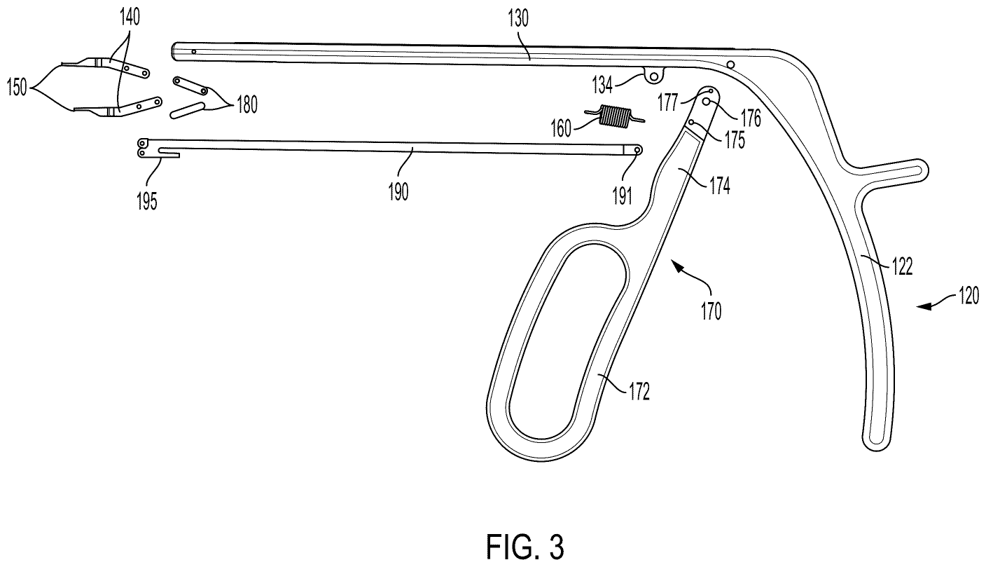

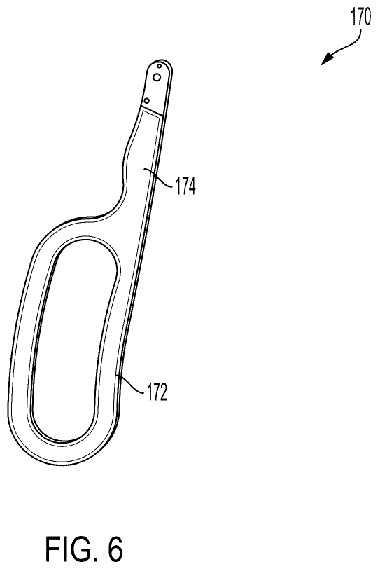

The actuator 170 is configured as best shown in FIGS. 1-3 and 6. The 170 actuator is pivotable relative to the handle 120 and movable between a neutral position and an operative position. The actuator includes an open, substantially ovoid gripping portion 172, which is sized to have an overall length sufficient to receive one or more of the fingers of a person's hand. In an alternative embodiment, the substantially ovoid gripping portion of the actuator can be a curved or straight gripping portion. As best shown in FIG. 3, an arm 174 extending from the substantially ovoid gripping portion 172 of the actuator 170 includes a first through hole 175 for receiving a proximal end of the biasing member 160, a second through hole 176 for pivotably connecting with the elongated frame 130 of the handle 120, and a third through hole 177 for pivotably connecting with the shaft 190. In other words, the arm 174 of the actuator 170 is pivotally connected to the elongated frame 130 and the shaft 190 near the junction of the elongated frame 130 with the curved gripping portion 122.

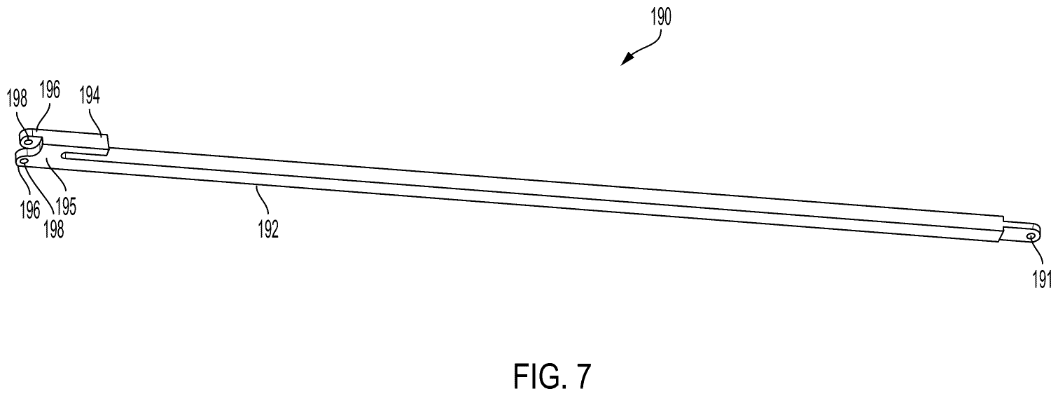

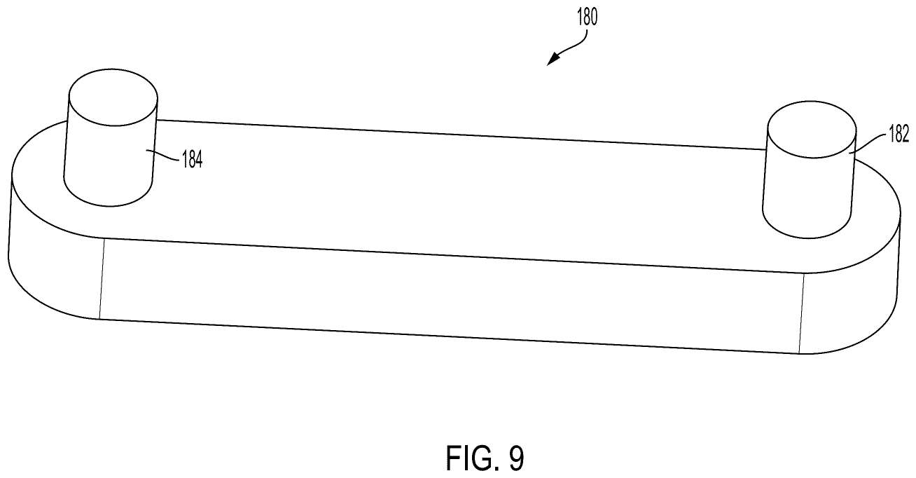

The shaft 190 is configured as best shown in FIGS. 2, 3, and 7. The shaft 190 is operatively connected to the actuator 170 and is elongated and sized to fit within the elongated frame 130. At its proximal end, the shaft 190 has an opening 191 configured to pivotably attach to the actuator 170 within the first recess 132 of the elongated frame via a third pivot 114. The distal portion of the shaft 190 includes the substantially U-shaped turn 195, wherein a first arm 192 of the U-shaped turn 192 runs for substantially the entire length of the shaft 190 and a second arm 194 of the U-shaped turn is substantially shorter, e.g., about 0.5 inches to about 2.0 inches, but can be more than 2 inches or less than 0.5 inches. The distal end of the shaft 190 includes a pair of lobes 196 pivotably connected to a pair of levers 180. That is, each lobe 196 has an opening 198 configured to receive a proximal pivot pin 182 of a lever 180 (FIG. 9).

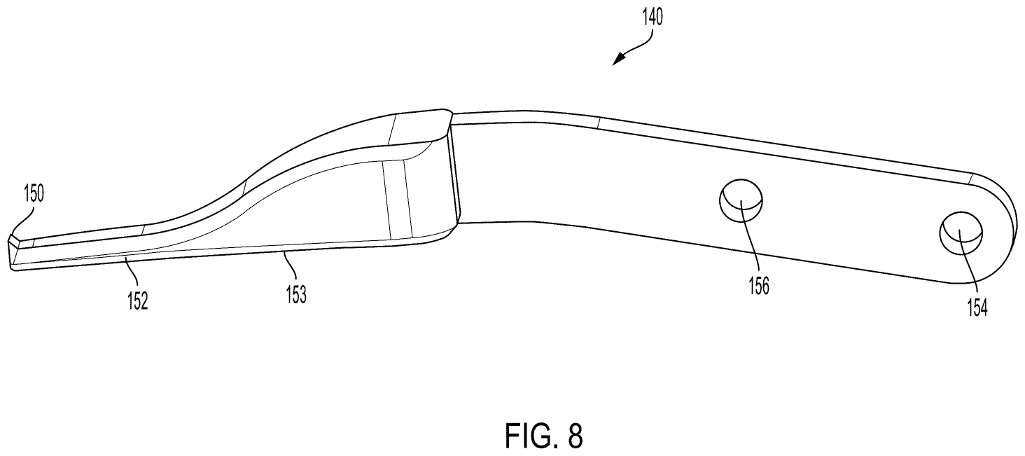

The levers 180 are best shown in FIGS. 1-3, 9 and 12. The pair of levers are pivotably connected to a distal end of the shaft 190 and pivotably connected to the pair of jaws 140. More particularly, each lever 180 has a distal end including a distal pivot pin 184 configured to pivotably connect to an opening 154 of a jaw (FIG. 8). The proximal end of each lever 180 includes the proximal pivot pin 182 for pivotably connecting to the opening 198 in a lobe 196 at the distal end of the shaft 190. The levers are arranged in an X-like configuration as shown in FIG. 12.

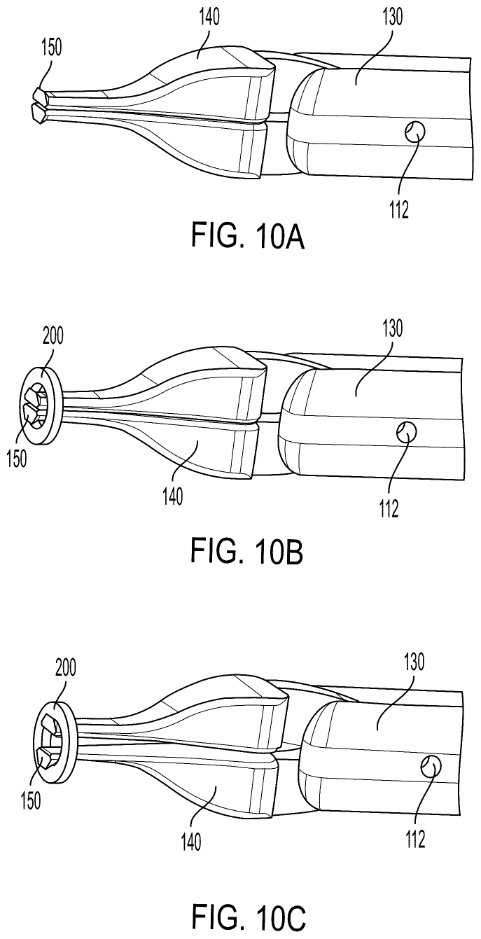

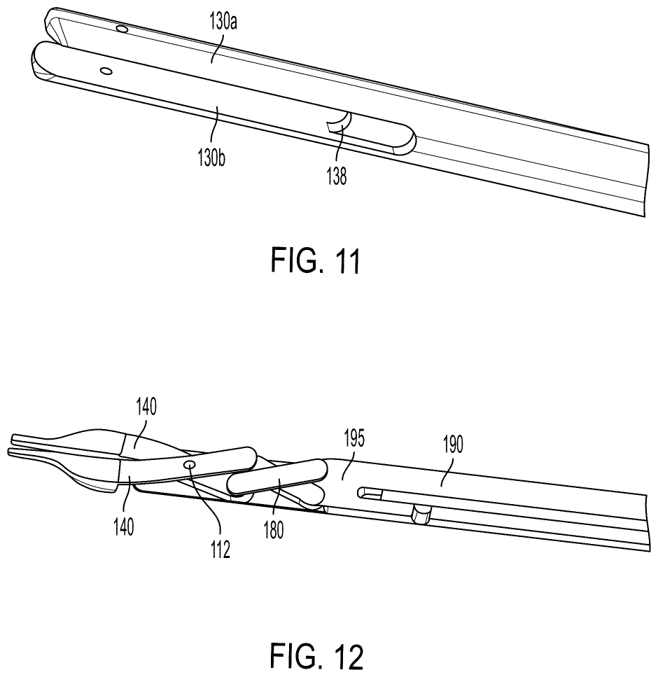

The pair of jaws 140 is configured as best shown in FIGS. 1-3, 8, 10A-10C and 12. The pair of jaws are operatively connected to the shaft 190. An exemplary structure of a jaw 140 is best shown in FIG. 8. It will be understood that the other jaw 140 is a mirror image of the jaw shown in FIG. 8. In this regard, the distal end of each jaw 140 includes an extension 152 sized and structured such that two jaws in a closed state fit through the open inner diameter of a surgical washer 200 (FIGS. 10B and 10C). As shown in FIG. 8, the upper jaw 140 includes a planar bottom side 153. The lower jaw 140 similarly includes a planar top side. The planar nature of the bottom side 153 of the upper jaw and the top side of the corresponding lower jaw enables the jaws to come into abutting contact with one another, whereby the contacting extensions presenting a minimal profile when inserted through the inner diameter of the surgical washer 200. The distal end of each extension 152 includes a radially outwardly extending flange 150. The proximal portions of the jaws 140 are configured to fit in the second recess 133, between the spaced apart arms 130a and 130b. The proximal portions of each jaw 140 includes a first through hole opening 154 to receive the distal pivot pin 184 of one of the levers 180. Additionally, each of the jaws includes a second through hole opening 156 for receiving the first pivot 112 for pivotably connecting the jaws 140 to the elongated frame 130, as shown in FIGS. 1, 2, 10A-10C and 12. When the actuator 170 is in the operative position the pair of jaws 140 move to an open state, as described in greater detail below.

An exemplary biasing member 160 applicable to the subject disclosure is shown in FIGS. 1-3. The biasing member 160 is operatively engaged with the actuator 170 and biases the actuator towards the neutral position. The biasing member can be a spring such as a coil spring, e.g., a tension spring, an elastomer, and the likeoperable to bias the actuator 170 to the neutral position. The biasing member 160 is connected at a distal end 164 thereof to the tab 134 of the elongated frame 130 near the elongated frame's junction with the curved gripping portion 122. Likewise, a proximal end 162 of the biasing member 160 is connected to through hole 175 in the arm 174 of the actuator 170. When the actuator 170 is squeezed by a user toward the gripping portion 122 of the handle 120 with a force sufficient to overcome the bias of the biasing member 160 the actuator is moved from its neutral to its operative state.

The arthroscopic washer extractor 100 of the subject disclosure is useful for removing a washer from a patient according to the steps, for example, shown in FIGS. 10A-10C. With the arthroscopic washer extractor 100 in a neutral state, the jaws 140 of the washer extractor are closed as shown in FIG. 10A. The jaws 140 in their neutral, closed state are extended though the central opening of the washer 200 until the outwardly extending flanges 150 pass completely through the inner diameter of the central opening of the washer 200, as shown in FIG. 10B. Next, the arthroscopic washer extractor is actuated. More particularly, the actuator 170 is retracted by a user to an operative position by applying squeezing force to the gripping portion 172 thereby causing the arm 174 of the actuator to push the shaft 190 distally. This, in turn, urges the levers 180 to pivot outwardly owing to its X-like configuration causing outward pivoting of the jaws 140 whereby their distal ends open until the outwardly extending flanges 150 of the jaws at least partially engage a distal surface of the washer 200 adjacent to the inner diameter of the washer, as shown in FIG. 10C. Once the washer is engaged by the outwardly extending flanges 150 of the jaws, it may be removed from the patient by withdrawing the arthroscopic washer extractor in its actuated or operative state.

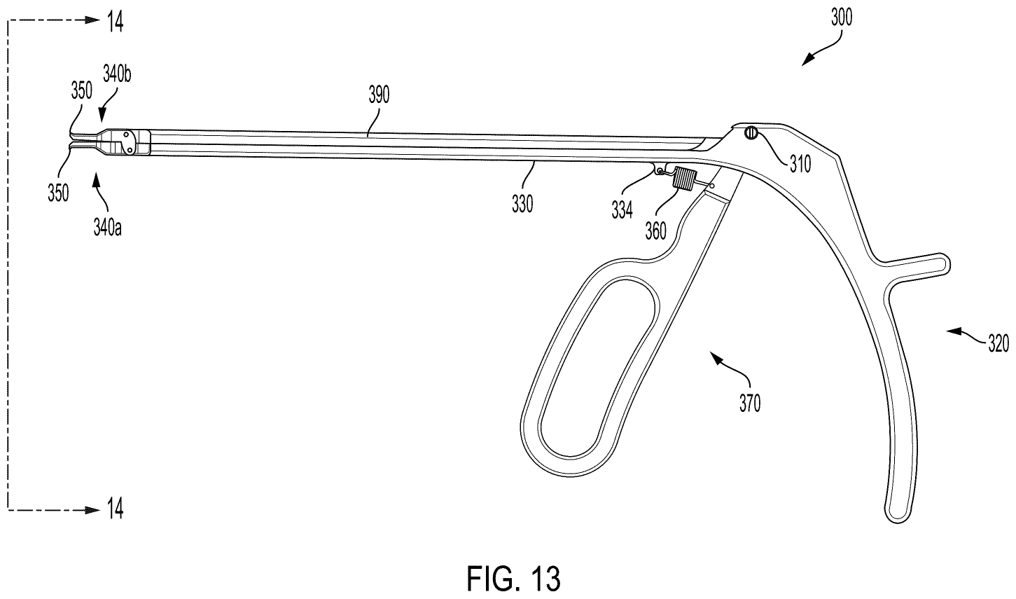

Referring to FIGS. 13-23 there is shown an arthroscopic washer extractor 300 constructed in accordance with a further exemplary embodiment of the subject disclosure. Only those features of arthroscopic washer extractor 300 that depart materially in structure and/or function from their counterparts of the arthroscopic washer extractor 100 will be described in detail.

FIG. 13 illustrates an exemplary embodiment of an arthroscopic washer extractor 300 according to the subject disclosure that comprises a handle 320, an actuator 370, a shaft 390, and a pair of jaws 340a and 340b. The distal ends of each of the pair of jaws 340a, 340b include radially outwardly extending flanges 350 similar in construction and function to their counterparts 150 described above.

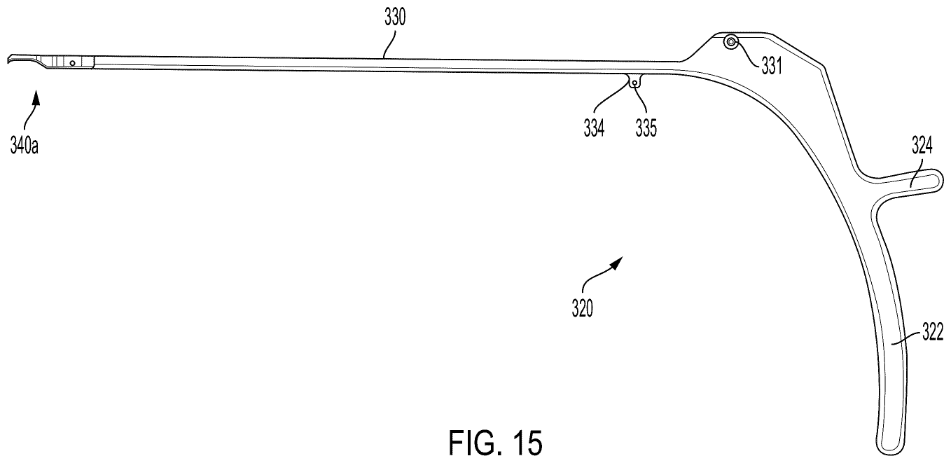

The handle 320 is configured as best shown in FIGS. 15-17. The handle 320 includes an elongated curved gripping portion 322 having a stop 324 extending posteriorly therefrom about an upper end of the gripping portion. Specifically, the stop extends about a portion one-third down from an uppermost end of the handle, e.g., about 1 to 2 inches from its uppermost end or about 2 to 4 inches from its bottom most end. The gripping portion 322 is sized to have an overall length sufficient for a palm of a person's hand to grasp, e.g., about 3 to 6 inches in length.

The handle 320 includes an elongated frame 330 extending therefrom. The elongated frame has a longitudinal axis extending substantially transverse to a longitudinal axis of the curved gripping portion 322. As shown in FIGS. 13 and 15, a proximal portion of the elongated frame adjacent to the elongated gripping portion includes a tab or protrusion 334 having a hole 335 (FIG. 15) to receive and connect to one end of a biasing member 360.

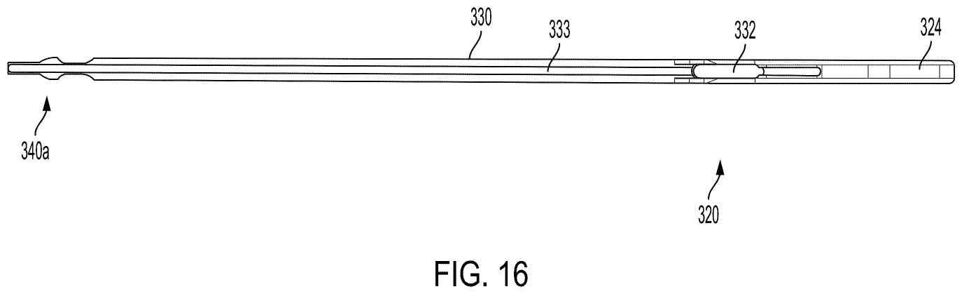

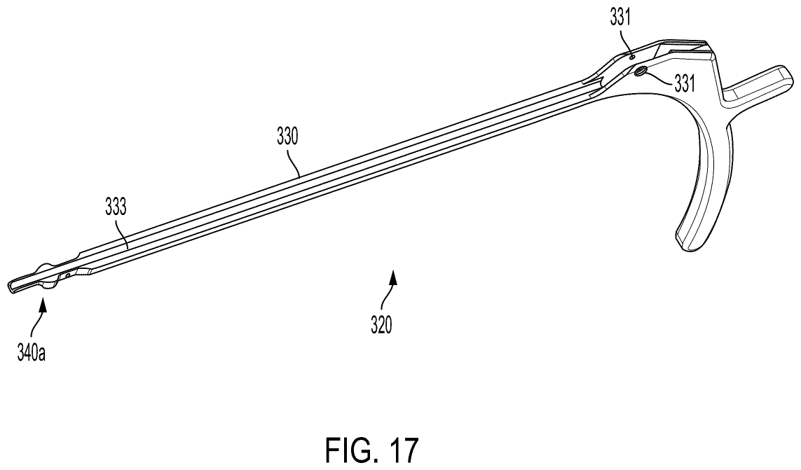

As shown in FIG. 16, the elongated frame 330 also includes a recess 332 about its proximal or posterior end. Referring to FIGS. 15 and 17, the elongated frame includes a pair of spaced apart aligned holes 331 about its proximal or posterior end for receiving a first pivot 310. By way the recess 332, the holes 331 and the first pivot 310, the proximal end of the elongated frame 330 of the handle 320 is configured to receive and pivotably mount the actuator 370. The first pivot 310 can be, e.g., a pin, screw or rivet, or the like.

As shown in FIGS. 16 and 17 an upper surface of the elongated frame 330 is provided with an elongated groove 333, the function of which is described in greater detail hereinafter. As best shown in FIG. 17, the elongated groove 333 is upwardly concave. Additionally, as shown most clearly in FIGS. 15-17, one of the pair of jaws, i.e., jaw 340a, is integrally connected to a distal end of the elongated frame, whereby the jaw 340a is stationary during operation of the arthroscopic washer extractor 300.

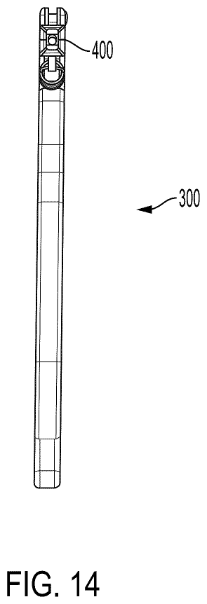

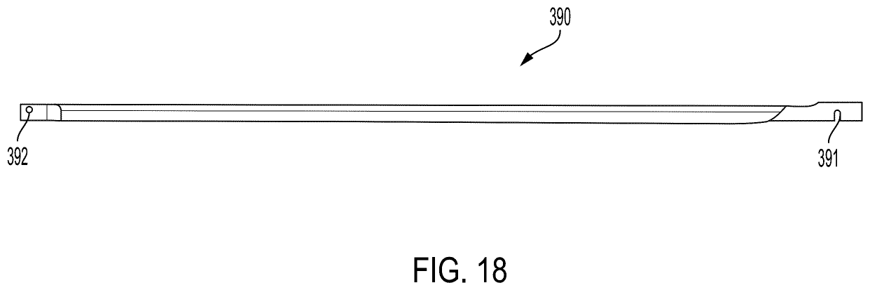

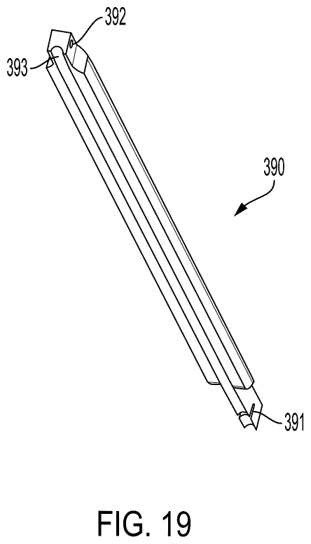

FIGS. 18 and 19 show an exemplary construction of the shaft 390. The shaft 390 is an elongated member having a proximal end including a vertical slot 391 for pivotably connecting the proximal end of the shaft to a hole in the actuator 370, as described below. The shaft 390 has a distal end including a transverse hole 392 for pivotably connecting the other of the pair of jaws 340b to the shaft, as described below. As best shown in FIG. 19, a lower surface of the shaft 390 is provided with an elongated groove 393. As best shown in FIG. 17, the elongated groove 393 is downwardly concave and lies in opposition to the elongated groove 333 of the elongated frame 330. By virtue of the opposed elongated grooves 333 and 393, the elongated frame 330 and the shaft 390 define a cannulation 400 (FIG. 14) for receiving an unillustrated guide wire or pin for guiding the distal ends of the jaws 340a, 340b through the central opening of the arthroscopic washer 200 during of a washer extraction procedure.

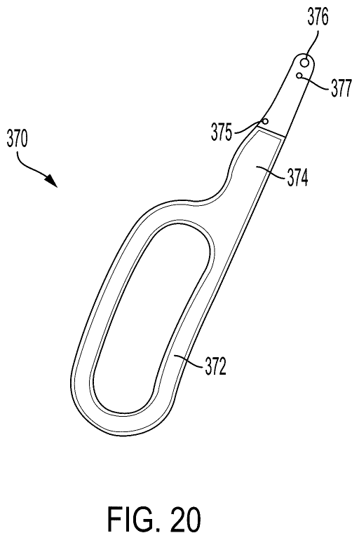

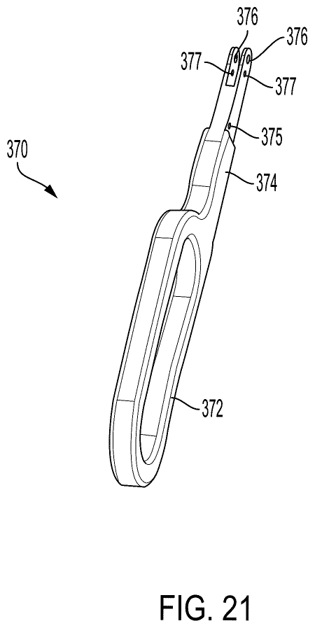

Referring to FIGS. 20 and 21, there is shown an exemplary construction of the actuator 370. The actuator 370 is pivotable relative to the handle 320 and movable between a neutral position and an operative position. The actuator includes an open, substantially ovoid gripping portion 372, which is sized to have an overall length sufficient to receive one or more of the fingers of a person's hand. In an alternative embodiment, the substantially ovoid gripping portion of the actuator can be a curved or straight gripping portion. An arm 374 extending from the substantially ovoid gripping portion 372 of the actuator 370 includes a first through hole 375 for receiving a proximal end of the biasing member 360, a second through hole 376 for pivotably connecting with the elongated frame 330 of the handle 220, and a third through hole 377 for pivotably connecting with the shaft 390. The second through hole 376 and the third through hole 377 are actually two pairs of aligned through holes provided on spaced apart lobes extending from the uppermost end of the arm 374 (FIG. 21). The lobes define a gap or slot for receiving the proximal end of the shaft 390 whereby an unillustrated pivot pin or the like pivotably connects the third through hole 377 with the vertical slot 391 provided in the proximal end of the shaft. Additionally, the relative positions of the second through hole 376 and third through hole 377 of the actuator 370 are reversed when compared to their counterparts in actuator 170 for reasons set forth below.

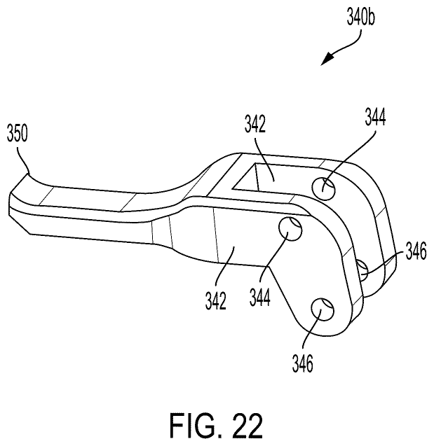

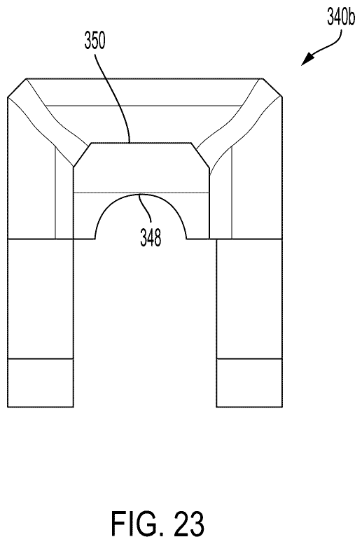

FIGS. 22 and 23 show an exemplary construction of the other of the pair of jaws, i.e., movable jaw 340b, in accordance with the subject disclosure. As noted above, at its distal end the movable jaw includes the radially outwardly extending flange 350. At its proximal end, the movable jaw includes a pair of spaced apart lobes 342 that are configured to straddle the distal ends of the elongated frame 330 and the shaft 390 (FIG. 1). The lobes 342 are provided with a pair of upper spaced apart and aligned holes 344 for pivotably connecting the movable jaw 340b to the shaft 390 by a suitable pivot, e.g., a pin, a rivet, a screw, or the like. In addition, the lobes 342 are provided with a pair of lower spaced apart and aligned holes 344 for pivotably connecting the movable jaw 340b to the elongated frame 300 by a suitable pivot, e.g., a pin, a rivet, a screw, or the like. As best shown in FIG. 23, the lower surface of the movable jaw 340b is provided with a downwardly concave groove 348 similar in cross-sectional shape to and axially aligned with the elongated groove 393 provided in the lower surface of the shaft 390 to provide a continuous cannulation 400 when the movable jaw is in its neutral, inoperative position.

The arthroscopic washer extractor 300 of the subject disclosure is also useful for removing a washer from a patient. With the arthroscopic washer extractor 300 in a neutral state, the movable jaw 340b of the washer extractor is closed against the fixed jaw 340a. The jaws 340a, 340b in their neutral, closed state are extended though the central opening of the washer 200 (possibly with the aid of a guide wire or pin received in the cannulation 400) until the outwardly extending flanges 350 pass completely through the inner diameter of the central opening of a washer 200, such as washer 200 shown in FIG. 10B. Next, the arthroscopic washer extractor 300 is actuated. More particularly, the actuator 370 is retracted by a user to an operative position by applying squeezing force to the gripping portion 372 thereby causing the arm 374 of the actuator to pull the shaft 390 proximally (by virtue of the pivoted connection of the shaft to the arm being below the pivoted connection of the pivoted connection of the elongated frame 330 to the arm). This, in turn, urges the movable jaw 340b to pivot outwardly away from the stationary lever 340a owing to the pivoted connection of the movable jaw to the stationary elongated frame 330, which does not translate, and the pivoted connection of the movable jaw to the movable shaft, which translates proximally and distally. In so doing, outward pivoting of the movable jaw 340b causes the radially outwardly extending flanges 350 of the jaws to at least partially engage a distal surface of the washer 200 adjacent to the inner diameter of the washer, similar to that shown in FIG. 10C. Once the washer is engaged by the outwardly extending flanges 350 of the jaws, it may be removed from the patient by withdrawing the arthroscopic washer extractor in its actuated or operative state.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the subject disclosure as defined by the appended claims.

1. An arthroscopic washer extractor comprising:
a handle;
an actuator pivotable relative to the handle and movable between a neutral position and an operative position;
a shaft operatively connected to the actuator;
a pair of jaws at least one of which is operatively connected to the shaft, wherein each of the pair of jaws includes a radially outwardly extending flange, and wherein the actuator is configured to move the pair of jaws to an open state.

2. The arthroscopic washer extractor of claim 1, further comprising a biasing member operatively engaged with the actuator and biasing the actuator towards the neutral position.

3. The arthroscopic washer extractor of claim 2, wherein the biasing member is a spring, a coil spring, a tension spring or an elastomer.

4. The arthroscopic washer extractor of claim 1, further comprising a pair of levers pivotably connected to a distal end of the shaft and pivotably connected to the pair of jaws.

5. The arthroscopic washer extractor of claim 4, wherein the distal end of the shaft is provided with a pair of lobes pivotably connected to the pair of levers.

6. The arthroscopic washer extractor of claim 1, wherein the handle includes an elongated frame.

7. The arthroscopic washer extractor of claim 6, wherein the elongated frame includes a pair of spaced-apart arms.

8. The arthroscopic washer extractor of claim 7, wherein the elongated frame includes a support beam extending between the pair of spaced-apart arms.

9. The arthroscopic washer extractor of claim 8, wherein the support beam forms boundaries of a first recess and a second recess provided in the elongated frame.

10. The arthroscopic washer extractor of claim 9, wherein the first recess is configured to receive and pivotably mount the actuator to a proximal end of the elongated frame.

11. The arthroscopic washer extractor of claim 9, wherein the second recess is configured to receive and pivotably mount the pair of jaws to a distal end of the elongated frame.

12. The arthroscopic washer extractor of claim 8, wherein the support beam has a notched nose at a distal end thereof.

13. The arthroscopic washer extractor of claim 12, wherein a distal end of the shaft includes a substantially U-shaped turn for receiving the notched nose of the support beam.

14. The arthroscopic washer extractor of claim 2, wherein an arm of the actuator includes a first through hole for receiving a proximal end of the biasing member.

15. The arthroscopic washer extractor of claim 14, wherein the arm of the actuator includes a second through hole for pivotably connecting with an elongated frame of the handle.

16. The arthroscopic washer extractor of claim 15, wherein the arm of the actuator includes a third through hole for pivotably connecting with the shaft.

17. The arthroscopic washer extractor of claim 6, wherein one of the pair of jaws is integrally connected to the elongated frame.

18. The arthroscopic washer extractor of claim 17, wherein the other of the pair of jaws is pivotably connected to the shaft.

19. The arthroscopic washer extractor of claim 18, wherein the other of the pair of jaws is pivotably connected to the elongated frame.

20. The arthroscopic washer extractor of claim 1, wherein the handle includes an elongated frame and wherein the elongated frame and the shaft define a cannulation.

21. The arthroscopic washer extractor of claim 20, wherein the cannulation is formed by opposed elongated grooves provided in the elongated frame and the shaft.

22. The arthroscopic washer extractor of claim 21, wherein the opposed elongated grooves are concave.

23. A method for grasping and removing a washer from a patient comprising:
introducing the arthroscopic washer extractor of claim 1 into a patient;
inserting the pair of jaws through a hole of a washer at least until the radially outwardly extending flanges of the jaws pass through the washer;
actuating the actuator to open the jaws whereby the radially outwardly extending flanges engage a distal face of the washer; and
pulling the arthroscopic washer extractor back toward a user with the opposing jaws in an open position.
